(19) United States
(12) Patent Application Publication
(10) Pub. No.: US 2026/0248427 A1
(43) Pub. Date: Aug. 27, 2026

(54) SYSTEM AND METHOD FOR CLASSIFYING EPISTEMIC COGNITION STATES OF A PERSON FROM EEG SIGNALS

(71) Applicant: Centre for Perceptual and Interactive Intelligence (CPII) Limited, Pak Shek Kok (CN)

(72) Inventor: Yuen Yan Chan, Pak Shek Kok (CN)

(21) Appl. No.: 19/065,425

(22) Filed: Feb. 27, 2025

(51) Int. Cl. A61B 5/16; A61B 5/00; A61B 5/374; G16H 50/70

(52) U.S. Cl. CPC A61B 5/165; A61B 5/374; A61B 5/7225; A61B 5/7267; G16H 50/70; A61B 2503/12

(57) ABSTRACT

A computer-implemented method for classifying epistemic cognition states of a person in educational contexts includes receiving an input dataset comprising EEG signals of a person, modifying the EEG signals by integrating educational domain knowledge to extract features related to epistemic cognition states, capturing local and long range temporal dependencies in the modified EEG signals, averaging across the temporal dimension the temporal dependencies in the modified EEG signals, classifying epistemic cognition states from the averaged temporal dependencies, outputting the classified epistemic cognition states.

### TECHNICAL FIELD

The present invention relates to a system and method for classifying epistemic cognition states of a person from EEG signals. In particular, the present invention relates to a system and method for classifying epistemic cognition states of a person from EEG signals in educational contexts.

### BACKGROUND

Epistemic cognition is an important area of research in educational studies. Epistemic cognition refers to the cognitive processes through which people understand, modify, and develop knowledge. It is rooted in the philosophical study of Epistemology and encompasses the nature of knowledge, including its sources, justification, and certainty.

Students' epistemic cognition significantly influences their approach to learning, problem-solving, and critical thinking. As learners navigate increasingly complex information landscapes, their epistemic beliefs shape how they critically engage with content, assess credibility, and construct their understanding. Research has consistently demonstrated the significance of epistemic cognition, showing that students with sophisticated epistemic beliefs exhibit deeper inquiry, stronger self-regulation, and better conceptual understanding.

Despite its importance, conventional educational research methods such as questionnaires and interviews often fail to capture the dynamic and situational nature of epistemic cognition.

### SUMMARY OF THE INVENTION

The present invention relates to a system and method for classifying epistemic states in humans. The system and method are particularly suited for classifying one or more epistemic cognition states of students. The system and method of the present invention classify one or more epistemic cognition states from EEG (electroencephalography) data of a student. The EEG data (i.e., EEG signals) are processed to identify one or more epistemic cognition states.

In accordance with a first aspect, there is provided a computer-implemented method for classifying epistemic cognition states of a person in educational contexts comprising:
receiving an input dataset comprising EEG signals of a person,
modifying the EEG signals by integrating educational domain knowledge to extract features related to epistemic cognition states,
capturing local and long-range temporal dependencies in the modified EEG signals,
averaging across the temporal dimension the temporal dependencies in the modified EEG signals,
classifying epistemic cognition states from the averaged temporal dependencies,
outputting the classified epistemic cognition states.

The method is advantageous because it can accurately identify epistemic cognition states. The method can accurately classify epistemic cognition states from EEG signals across various educational contexts or educational settings. The method is advantageous and provides a more accurate output because the method modifies the EEG signals by integrating educational domain specific knowledge.

In one example the step of modifying the EEG signals by integrating educational domain knowledge comprising:
bandpass filtering the EEG signals to isolate one or more predefined frequency bands,
spatial filtering the EEG signals to select channels associated with epistemic cognition,
temporal filtering the EEG signals to focus on signals with specific epistemic event windows.

In one example the step of modifying the EEG signals comprising encoding categorical data as embeddings to incorporate contextual information into the modified EEG signals.

In one example the one or more predefined frequency bands have a frequency of between 0.01 Hz-30 Hz.

In one example, the predefined frequency band may be between 1 Hz-20 Hz.

In one example the one or more predefined frequency bands are delta, theta, alpha and beta frequency bands. In this example, delta, theta, alpha and beta frequency bands may correspond to frequency bands in EEG signals.

In one example, the EEG signals may be filtered by applying one or more bandpass filters to filter the EEG signals based on delta, theta, alpha and beta frequency bands. The EEG signals may be filtered into multiple frequency bands.

In one example the channels may be channels associated with epistemic cognition comprise frontal region channels.

In one example spatial filtering selects channels F3, F4, F7, F8, AF3 and AF4, wherein channels F3, F4, F7, F8, AF3 and AF4 are associated with epistemic cognition.

In one example modifying the EEG signals by integrating educational domain knowledge comprising: preprocessing the EEG signals, by applying independent component analysis to remove artefacts and normalize the EEG signals across channels to output two-dimensional multi-channel time series data.

In one example modifying the EEG signals by integrating educational domain knowledge comprising:
projecting the preprocessed EEG signals into a high dimensional feature space,
concatenating the projected preprocessed EEG signals with categorical variable embeddings, wherein the embeddings comprise integrating educational data and EEG signals into a unified representation, and;
segmenting the data into fixed length epochs by applying a sliding window technique.

In one example the method comprising adding sinusoidal positional encodings into the embedded features or the fixed length epochs to retain temporal context based on unique positional information.

In one example the step of modifying the EEG signals by integrating educational domain knowledge results in a C×T×d_{model} tensor, where each time step and each channel comprises signal features and positional context, and wherein C represents the number of channels in the EEG signals, T represents the total time steps and d_{model} represents a unified representation that integrates EEG signals and educational data.

In one example the method comprising iteratively refining feature representation over a predefined number of iterations by attending to temporal patterns and enhancing contextual relationships.

In accordance with a second aspect, there is provided a machine learning model for classifying epistemic cognition states of a person in educational contexts, for use in the method of the statements described in the first aspect comprising:
an input representation block configured to receive raw EEG signals and modify the EEG signals by integrating educational domain knowledge to extract features related to epistemic cognition states,
a transformer encoder block operatively coupled to the input representation block, wherein the transformer encoder block is positioned downstream to the input representation block,
wherein the transformer encoder block configured to capture local and long-range temporal dependencies in the modified EEG signals,
   a global average pooling block operatively coupled to the transformer encoder block, wherein the global average pooling block being positioned downstream of the transformer encoder block,
   wherein the global average pooling block configured to average across the temporal dimension the temporal dependencies in the modified EEG signals,
   a classification block operatively coupled to the global average pooling block, the classification block positioned downstream of the global average pooling block,
wherein the classification block is configured to classify epistemic cognition states from the averaged temporal dependencies and output the classified epistemic cognition states.

In accordance with a further aspect, there is provided a machine learning model for classifying epistemic cognition states of a person in educational contexts, comprising:
an input representation block, the input representation block configured to transform raw EEG signals by incorporating educational domain knowledge to extract features for epistemic studies,
a transformer encoder block, the transformer encoder block positioned after the input representation block and arranged in communication with the input representation block,
the transformer encoder block is adapted to receive the transformed EEG signals from the input representation block,
wherein the transformer encoder block configured to implement a transformer encoder that is adapted to capture local and long-range temporal dependencies in the EEG signals, the transformer encoder block is configured to refine feature representations over a plurality of iterations,
a global average pooling block located after the transformer encoder block and arranged in communication with the transformer encoder block,
wherein the global average pooling block is configured to reduce sequence length by averaging the output of the transformer encoder block across a temporal dimension into a single value per feature channel,
a classification block located after the global average pooling block, the classification block is arranged in communication with the global average pooling block,
wherein the classification block is configured to translate features from the global average pooling block into predictions of epistemic cognition states, and;
wherein the classification block is configured to classify epistemic cognition states based on the predictions and output the classified epistemic cognition states.

In one example, the machine learning model is a transformer-based EEG classifier,
wherein the input representation block comprising:
   a data acquisition module configured to preprocess raw EEG signals using independent component analysis to remove artefacts and normalize the EEG signals across multiple channels, the data acquisition module configured to output a 2D multi-channel time series,
   educational context preprocessing (ECP) module operatively coupled to the data acquisition module, the ECP module configured to:
   receive the output of the data acquisition module,
   apply a bandpass filter to the preprocessed EEG signals from the data acquisition module to isolate one or more predefined frequency bands, wherein the one or more predefined frequency bands are delta, theta, alpha and beta frequency bands,
   spatially filter to select channels associated with epistemic cognitive processing, and;
   temporal filter to focus on EEG signals within one or more epistemic event windows,
   a feature embedding module operatively coupled to the ECP module, the feature embedding module configured to:
   project the preprocessed EEG signals into a high dimensional feature space,
   concatenate the projected signals with categorical variable embeddings thereby integrating educational data and EEG signals into a unified representation,
   a positional encoding module operatively coupled to feature embedding module, the positional encoding module configured to add sinusoidal positional encodings to the embedded features,
wherein the transformer encoder block comprising:
   a multi head attention layer configured to compute contextual relationships between all-time steps in the sequence,
   a feed forward network (FFN) component operatively coupled to the multi head attention layer, the FFN comprising a plurality of convolution layers and at least one dropout layer,
   wherein the FFN configured to refine the attention output through the plurality of convolution layers and the one dropout layer,
wherein the classification block comprises:
   a multi-layer perceptron (MLP) that comprises a plurality of fully connected layers, wherein the MLP is configured to receive a feature vector from the transformer encoder block and process the feature vector through the connected layers by with ReLU activation to capture non-linear relationships,
   a dropout regularization module that is configured to deactivate one or more neurones in the MLP to prevent overfitting, and;
   an output layer that is configured to output a probability score for each class, wherein the probability score indicating the classification of a epistemic cognition state.

In accordance with a further aspect, there is provided a system for classifying epistemic cognition states of a person in educational contexts, comprising:
a computing apparatus comprising a processor and a memory unit being operatively coupled to each other,
the computing apparatus is configured to:
   receive raw EEG signals,
   process the raw EEG signals by applying a transformer-based EEG classifier to classify epistemic cognition states within the EEG signals,
   wherein the transformer-based EEG classifier is configured to integrate educational domain knowledge into the raw EEG signals as part of the processing, and
   output the classified epistemic cognition states.

In one example the computing apparatus is configured to:
receive an input dataset comprising EEG signals of a person,
modify the EEG signals by integrating educational domain knowledge to extract features related to epistemic cognition states,
capture local and long-range temporal dependencies in the modified EEG signals,
average across the temporal dimension the temporal dependencies in the modified EEG signals,
classify epistemic cognition states from the averaged temporal dependencies,
output the classified epistemic cognition states.

In one example when the transformer-based EEG classifier executed by the processor, the computing apparatus is further configured to:
bandpass filter the EEG signals to isolate one or more predefined frequency bands,
spatial filter the EEG signals to select channels associated with epistemic cognition,
temporal filter the EEG signals to focus on signals with specific epistemic event windows.

In one example the one or more predefined frequency bands are delta, theta, alpha and beta frequency bands, the channels associated with epistemic cognition comprise frontal region channels and; spatial filtering selects channels F3, F4, F7, F8, AF3 and AF4, wherein channels F3, F4, F7, F8, AF3 and AF4 are associated with epistemic cognition.

In one example the transformer-based EEG classifier comprises:
an input representation block configured to receive raw EEG signals and modify the EEG signals by integrating educational domain knowledge to extract features related to epistemic cognition states,
a transformer encoder block operatively coupled to the input representation block, wherein the transformer encoder block is positioned downstream to the input representation block,
wherein the transformer encoder block configured to capture local and long-range temporal dependencies in the modified EEG signals,
a global average pooling block operatively coupled to the transformer encoder block, wherein the global average pooling block being positioned downstream of the transformer encoder block,
wherein the global average pooling block configured to average across the temporal dimension the temporal dependencies in the modified EEG signals,
a classification block operatively coupled to the global average pooling block, the classification block positioned downstream of the global average pooling block,
wherein the classification block is configured to classify epistemic cognition states from the averaged temporal dependencies and output the classified epistemic cognition states.

In one example, the system may comprise a user interface e.g., a display or touchscreen that is configured present the classified epistemic cognition states.

The presented epistemic cognition states can be used by educators to help educators identify states such as “understanding” or “confusion” in real time, allowing educators to tailor their teaching techniques. Additionally, the classified or identified epistemic cognitive states can be presented to educators and allow educators to identify students that may be struggling with comprehension or learning allowing for more personalized teaching. The system is advantageous because classifying epistemic cognition states can provide actionable insights that can be used to re-shape education and teaching strategies providing improved teaching.

In accordance with a further aspect, there is provided a computer-implemented method of training a machine-learning model for classifying epistemic cognition states of a person in educational contexts, in particular the machine-learning model as described herein comprising:
receiving an input training dataset comprising preprocessed EEG signals, dividing the input training dataset into a training set and test set according to an 80% and 20% ratio,
utilizing an Adam optimizer with a learning rate of 1×10⁻⁴ and batch size of 32 to train the machine learning model, wherein the machine learning model may be a transformer-based EEG classifier,
a validation set is randomly extracted from the training set to assess model convergence,
evaluating the model performance by processing the test samples.

In one example, training dataset may comprise one or more publicly available EEG datasets collected during epistemic cognitive tasks. The dataset or datasets may encompass diverse educational contexts and participant characteristics.

In accordance with a further aspect, there is provided a data processing apparatus for classifying epistemic cognition states of a person in educational contexts comprising a hardware processor that is configured to carry out the method described in accordance with a first aspect or execute a method for classifying epistemic cognition states of a person in educational contexts as described herein.

In accordance with a further aspect, there is provided a computer program comprising instructions, which when the program is executed by a computer, or a processor of a computer (or computing apparatus) causes the computer to carry out the method for classifying epistemic cognition states of a person in educational contexts as described herein.

In accordance with a further aspect, there is provided a non-transitory computer readable medium comprising instructions which, when executed by a computer or a processor of a computer, causes the computer to carry out the method for classifying epistemic cognition states of a person in educational contexts as described herein.

The term “machine learning model” may also be referred to as deep learning models, modules, systems or architectures or other AI based networks or neural network architecture, for example, and without limitations, transformers.

The term “comprising” (and its grammatical variations) as used herein are used in the inclusive sense of “having” or “including” and not in the sense of “consisting only of”.

It is to be understood that, if any prior art information is referred to herein, such reference does not constitute an admission that the information forms a part of the common general knowledge in the art.

### BRIEF DESCRIPTION OF THE DRAWINGS

Embodiments of the present invention will now be described, by way of example, with reference to the accompanying drawings in which:

FIG. 1 illustrates a diagram of a system for classifying epistemic cognition states of a person from EEG signals in educational contexts.

FIG. 2 illustrates a schematic diagram of a computing apparatus that implements a transformer-based EEG classifier in the system of FIG. 1.

FIG. 3 illustrates a flow chart of a method for classifying epistemic cognition states of a person from EEG signals in educational contexts.

FIG. 4 illustrates an example of a transformer architecture (i.e., a transformer-based EEG classifier) used as part of the system of FIG. 1.

FIG. 5 illustrates the overview of the datasets used in the experiments by the inventors.

FIG. 6 illustrates a table of test results of the transformer model with the first set of data.

FIG. 7 illustrates a table of test results of the transformer model with the second set of data.

FIG. 8 illustrates a table of test results of the transformer model with the third set of data.

FIG. 9 illustrates a graph showing the performance comparison of the transformer model of FIG. 4 and a modified model in a first ablation experiment.

FIG. 10 illustrates a graph showing the performance comparison of the transformer model of FIG. 4 and a modified model in a second ablation experiment.

FIG. 11 illustrates a graph showing the performance comparison of the transformer model of FIG. 4 and a modified model in a third ablation experiment.

FIG. 12 illustrates a graph showing comparison of the transformer model of FIG. 4 and a modified model comparing categorical embedding of gender.

FIG. 13 illustrates a graph showing comparison of the transformer model of FIG. 4 and a modified model comparing categorical embedding of lesson ID.

FIG. 14 illustrates a table comparing the performance of the transformer model of FIG. 4 and other state of the art EEG classification models for cognitive processing.

### DETAILED DESCRIPTION OF THE PREFERRED EMBODIMENTS

Epistemic cognition in humans refers to cognition and cognitive processes related to knowledge matters such as knowing and understanding. In short, it is the study of thinking about knowledge and knowing. It encompasses how individuals evaluate, justify, and regulate their beliefs and knowledge in different contexts. This process is foundational to learning and decision-making, influencing how individuals interpret information, draw conclusions, and adapt their understanding to new situations. Epistemic cognition is an advanced level of human cognitive processing, which is above cognition (e.g., perceiving through reading, writing, listening, and memorizing) and metacognition (e.g., regulating one's own cognitive processes). This makes epistemic cognition particularly significant in educational settings, as it impacts students' engagement with content, critical thinking skills, and ability to synthesize information across domains.

Since epistemic cognition fundamentally involves cognitive processes, it can be scientifically examined through the measurement of brain activity and related neural mechanisms. Neuroscience studies have increasingly contributed to our understanding of epistemic cognition and its underlying neural correlates. For example, findings from fMRI studies reveal that epistemic curiosity is modulated by prior knowledge and surprise, with activity in the rostrolateral prefrontal cortex correlating with curiosity levels and the ventral striatum engaged during the relief of curiosity when knowledge delivery is unpredictable. Furthermore, fMRI studies on epistemic judgment activate the inferior frontal gyrus (IFG) and middle frontal gyrus (MFG), suggesting their involvement in semantic control and decision-making processes.

EEG-based research has also revealed distinguishable spatial, temporal, and spectral patterns in diverse epistemic states. The present invention relates to a system and method for classifying epistemic cognition states of a person from EEG signals in educational contexts. The system utilises a transformer model for classifying students' epistemic cognition using EEG data. By incorporating education domain-specific considerations into a transformer based classifier, the system and method for classifying epistemic states provide a robust tool for real-time assessment of epistemic cognition during students' learning activities.

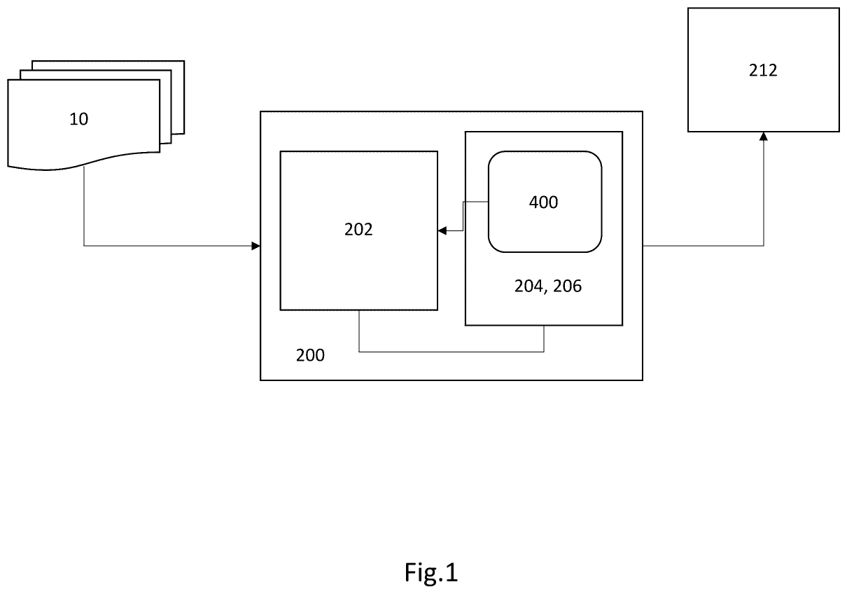

FIG. 1 illustrates example a system 100 for classifying epistemic cognition states of a person in educational contexts. The system 100 comprising a computing apparatus 200 comprising a processor 202 and a memory unit 204, 206 being operatively coupled to each other, wherein the computing apparatus 200 is configured to: receive raw EEG signals 10, process the raw EEG signals by applying a transformer based EEG classifier 400 to classify epistemic cognition states within the EEG signals, wherein the transformer based EEG classifier is configured to integrate educational domain knowledge into the raw EEG signals as part of the processing, and output the classified epistemic cognition states. The classifier 400 may be stored in a memory and executed by the processor 202.

The system 100 may comprise a display 212 that may be configured to present the classified epistemic cognition states. The system 100 may be configured to process EEG signals from multiple persons e.g., students to classify (i.e., identify) epistemic cognition states during various educational contexts. The system 100 may be used by an authorized person e.g., an educator or a government department or a research organization to classify epistemic cognition states of students while in one or more education settings. The classified cognition states can allow an authorized person e.g., a teacher to track students and identify students that are struggling to learn based on the classified epistemic cognitive states.

The computing apparatus 200 may be configured to receive an input dataset comprising EEG signals of a person. The EEG signals may be measured by a plurality of electrodes. The computing apparatus 200 is further configured to modify the EEG signals by integrating educational domain knowledge to extract features related to epistemic cognition states and capture local and long-range temporal dependencies in the modified EEG signals. The computing apparatus 200 is configured to average across the temporal dimension the temporal dependencies in the modified EEG signals and classify epistemic cognition states from the averaged temporal dependencies. The computing apparatus 200 may be further configured to output the classified epistemic cognition states.

In this example embodiment, the transformer-based EEG classifier 400 may be a type of machine learning model that may be used for real time identification of epistemic cognition states or may perform classification on pre-measured data. The transformer-based EEG classifier 400 may be stored in and executed by a computing apparatus 200 (or a computer).

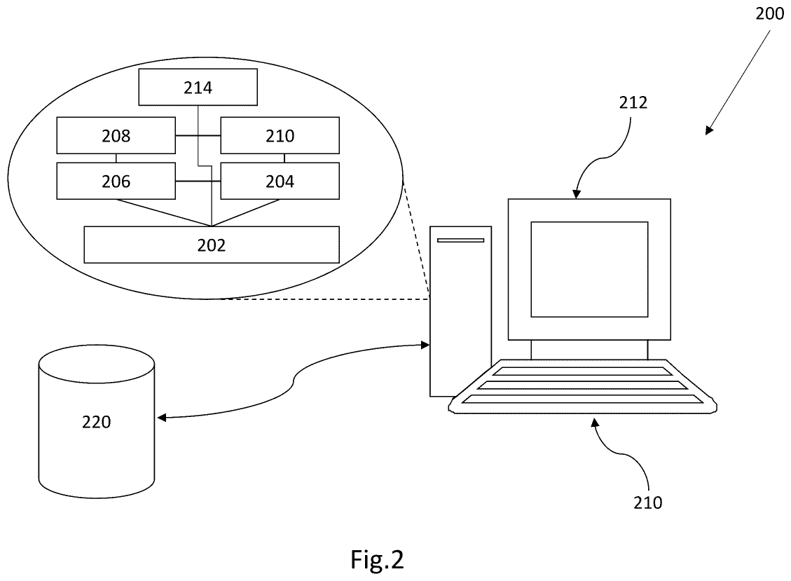

FIG. 2 illustrates a schematic diagram of a computing apparatus 200 which is arranged to be implemented as an example embodiment of a system for classifying epistemic cognition states of a person in educational contexts. Referring to FIG. 2, the computing apparatus 200 comprises components necessary to receive, store and execute appropriate computer instructions.

The components may include a processor 202 (i.e., a processing unit), including Central Processing Unit (CPU), Math Co-Processing Unit (Math Processor), Graphic Processing Unit (GPUs) or Tensor processing unit (TPUs) for tensor or multi-dimensional array calculations or manipulation operations, read-only memory (ROM) 204, random access memory (RAM) 206, and input/output devices such as disk drives 208, input devices 210 such as an Ethernet port, a USB port, etc.

Optionally, the computing apparatus 200 may comprise a display 212 such as a liquid crystal display, a light emitting display or any other suitable display. The display 212 may be integrated into the computing apparatus 200 or may be remote from the computing apparatus 200. The computing apparatus 200 may include instructions that may be included in ROM 204, RAM 206 or disk drives 208 and may be executed by the processor 202. There may be provided a plurality of communication links 214 which may variously connect to one or more other computing devices such as a server, personal computers, terminals, wireless or handheld computing devices, Internet of Things (IoT) devices, smart devices, edge computing devices. At least one of a plurality of communications link may be connected to an external computing network through a telephone line or other type of communications link. The communications link may allow the computing apparatus 200 to communicate via any suitable communication network such as for example a cellular network or Wi-Fi or other communication networks.

The computing apparatus 200 may include storage devices such as a disk drive 208 which may encompass solid state drives, hard disk drives, optical drives, magnetic tape drives or remote or cloud-based storage devices. The computing apparatus 200 may use a single disk drive or multiple disk drives, or a remote storage service. The computing apparatus 200 may also have a suitable operating system which resides on the disk drive or in the ROM of the computing apparatus 200.

The computing apparatus may further comprise one or more databases adapted to store one or more pieces of data. In one example, the computing apparatus 200 may include a EEG signal database 220. The EEG signal database 220 may store one or more training datasets. The EEG signal database 220 may optionally store measured EEG signals from one or more students in various educational contexts.

The computing apparatus 200 may also provide the necessary computational capabilities to operate or to interface with a machine learning network, such as a neural networks, to provide various functions and outputs. The neural network may be implemented locally, or it may also be accessible or partially accessible via a server or cloud-based service. The machine learning network may also be untrained, partially trained or fully trained, and/or may also be retrained, adapted or updated over time. The computing apparatus may comprise one or more GPUs being operatively coupled to the CPU (i.e., processor). The computing apparatus may comprise additional hardware elements operatively coupled to the CPU and/or the GPU to provide the computing apparatus components needed to implement a machine learning network or machine learning model. The learning network or model may be stored in a memory unit e.g., ROM.

The computing apparatus 200 may be implemented by any computing architecture, including portable computers, tablet computers, stand-alone Personal Computers (PCs), smart devices, Internet of Things (IOT) devices, edge computing devices, client/server architecture, “dumb” terminal/mainframe architecture, cloud-computing based architecture, or any other appropriate architecture. The computing apparatus 200 may further be implemented by smartphones or tablets or smart watches or other portable devices. The computing apparatus 200 (i.e., computing device or computer or server) may be appropriately programmed to implement a transformer-based EEG classifier 400, when executed causes the computing apparatus 200 to classify epistemic cognition states in educational contexts. In another example, the computing apparatus 200 may be implemented on a single computing device e.g., as shown in FIG. 2. Alternatively, the computing apparatus 200 may be implemented on edge computing devices or may be implemented as a distributed computing system.

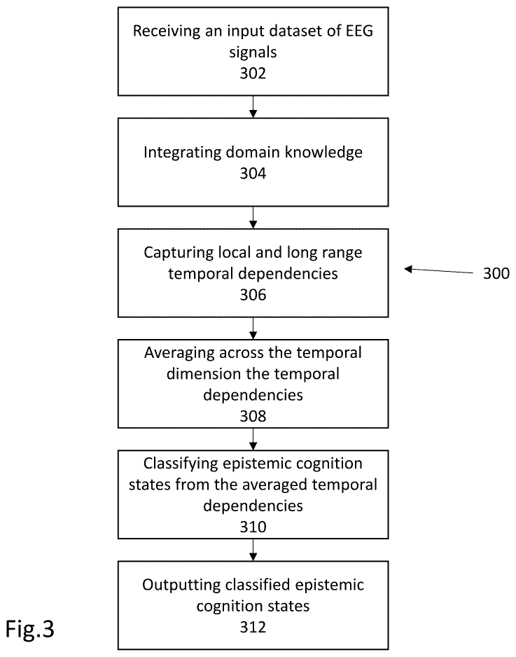

FIG. 3 illustrates an example of a computer-implemented method 300 for classifying epistemic cognition states of a person in educational contexts. The method 300 may commence at step 302. Step 302 comprises receiving an input dataset comprising EEG signals of a person. Step 302 may comprise a plurality of EEG signals from multiple persons (e.g., students). Step 304 comprises modifying the EEG signals by integrating educational domain knowledge to extract features related to epistemic cognition states.

Step 306 comprises capturing local and long-range temporal dependencies in the modified EEG signals. Step 308 comprises averaging across the temporal dimension the temporal dependencies in the modified EEG signals. Step 310 comprises classifying epistemic cognition states from the averaged temporal dependencies. Step 312 comprises outputting the classified epistemic cognition states. Step 312 may be optional. The classified epistemic cognition states may be presented on a display 212.

The method is advantageous because it can accurately identify epistemic cognition states. The method can accurately classify epistemic cognition states from EEG signals across various educational contexts or educational settings. The method is advantageous and provides a more accurate output because the method modifies the EEG signals by integrating educational domain specific knowledge.

The step of modifying the EEG signals by integrating educational domain knowledge may include bandpass filtering the EEG signals to isolate one or more predefined frequency bands. Integrating educational domain knowledge may include spatial filtering the EEG signals to select channels associated with epistemic cognition. Integrating educational domain knowledge may include temporal filtering the EEG signals to focus on signals with specific epistemic event windows.

In one example the step of modifying the EEG signals comprises encoding categorical data as embeddings to incorporate contextual information into the modified EEG signals. In one example the one or more predefined frequency bands have a frequency of between 0.01 Hz-30 Hz. More preferably the predefined frequency band may be between 1 Hz-20 Hz. In one example the one or more predefined frequency bands are delta, theta, alpha and beta frequency bands. In this example, delta, theta, alpha and beta frequency bands may correspond to frequency bands in EEG signals.

In one example the channels may be channels associated with epistemic cognition comprise frontal region channels.

In one example spatial filtering selects channels F3, F4, F7, F8, AF3 and AF4, wherein channels F3, F4, F7, F8, AF3 and AF4 are associated with epistemic cognition.

In one example modifying the EEG signals by integrating educational domain knowledge comprising: preprocessing the EEG signals, by applying independent component analysis to remove artefacts and normalize the EEG signals across channels to output two-dimensional multi-channel time series data.

The method 300 may be executed by the computing apparatus 200. The method 300 may be embodied as readable and executable instructions. The instructions may be stored in a non-transitory computer readable medium e.g., a memory unit. The processor 202 may be configured to execute the readable and executable instructions. The computing apparatus 200 is configured to execute the steps of method 300 when the processor 202 executes the readable and executable instructions.

A transformer is a type of a machine learning model, in particular a deep learning model that comprises an encoder-decoder structure but does not rely on recurrence and convolutions to generate an output. The transformer provides a highly effective mechanism for modelling sequential data. The encoder, on the left half of the Transformer architecture, is configured to map an input sequence to a sequence of continuous representations, which is then fed into a decoder. The decoder, on the right half of the architecture, receives the output of the encoder together with the decoder output at the previous time step to generate an output sequence. Transformers may include a self-attention mechanism, which enables the modelling of long-range dependencies and contextual relationships. This makes transformers particularly suitable for tasks that require understanding complex patterns over time, such as EEG signals. Unlike recurrent neural networks (RNNs), which process data sequentially, transformers operate in parallel, offering improved computational efficiency and scalability.

Transformers have emerged as a powerful tool for EEG signal analysis, too. This is because of their ability to address challenges such as non-stationarity, noise, and high inter-subject variability inherent in brain data.

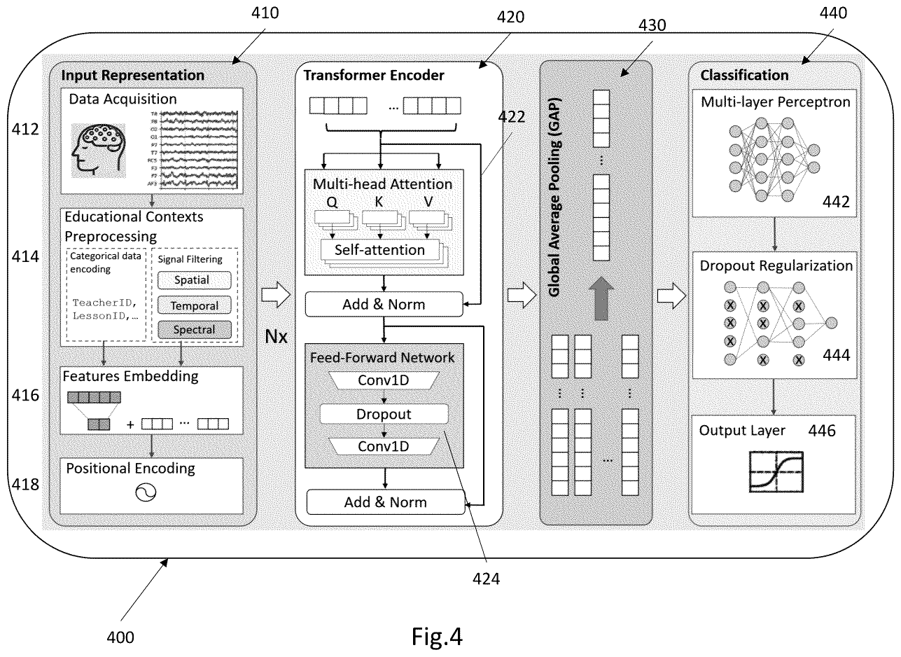

FIG. 4 illustrates an example of a transformer 400 architecture used as part of the system 200 for classifying epistemic cognition states of a person from EEG signals in educational contexts. The method steps of method 300 may be executed by the transformer 400. The transformer architecture 400 includes a education contexts processing block or module that integrates educational domain knowledge to extract features related to epistemic cognition states.

The transformer 400 incorporates educational contexts preprocessing layer (ECP) 414. The transformer 400 (i.e., a transformer-based EEG classifier) is configured for processing and classifying EEG signals collected during epistemic activities in educational contexts. By combining the transformer's capabilities for handling sequential data with preprocessing tailored to educational data inputs, this architecture optimally addresses the unique challenges posed by EEG data in these settings.

Referring to FIG. 4, the transformer 500 (i.e., transformer-based EEG classifier) comprises an input representation block 410. The input representation block 410 is configured to transform raw EEG signals by incorporating educational domain knowledge to extract features for epistemic studies. The input representation block 410 transforms raw EEG signals into a structured format for processing. It performs preprocessing, embedding, and positional encoding to represent the temporal, spatial, and spectral characteristics of EEG data effectively. EEG channels follow the 10-20 system for consistent spatial mapping of brain activity across regions.

The input representation block 410 comprises an data acquisition module 412, an educational context preprocessing (ECP) module 414, a feature embedding module 416 and a positional encoding module 418. The positional encoding module 418 may be an optional feature.

In the data acquisition module 412 Raw EEG signals are pre-processed using Independent Component Analysis (ICA) for artifact removal and normalized across channels. The resulting 2D multi-channel time-series data is represented as C×T, where C is the number of channels and T is the total time steps. The data acquisition module 412 configured to output a 2D multi-channel time series.

The ECP module 414 is operatively coupled to the data acquisition module 412. The ECP module 414 is configured to receive the output of the data acquisition module and apply one or more types of filters. For example, the ECP module 414 is configured to apply a bandpass filter to the preprocessed EEG signals from the data acquisition module to isolate one or more predefined frequency bands, wherein the one or more predefined frequency bands are delta, theta, alpha and beta frequency bands. The ECP module 414 is further configured to apply a spatially filter to select channels associated with epistemic cognitive processing. Finally, the ECP module 414 is configured to apply temporal filter to focus on EEG signals within one or more epistemic event windows.

The ECP module 414 is tailored to process EEG data for educational analysis, ensuring optimal feature extraction for epistemic studies. This stage involves bandpass filtering to isolate relevant frequency bands (e.g., 1-20 Hz), spatial filtering to select channels associated with epistemic cognitive processing (e.g., frontal region channels), and temporal filtering to focus on signals within specific epistemic event windows. Additionally, categorical data such as lesson ID are encoded as embeddings to incorporate contextual information.

In one example the one or more predefined frequency bands have a frequency of between 0.01 Hz-30 Hz. Preferably the predefined frequency band may be between 1 Hz-20 Hz. In one example the one or more predefined frequency bands are delta, theta, alpha and beta frequency bands. In this example, delta, theta, alpha and beta frequency bands may correspond to frequency bands in EEG signals.

In one example, the EEG signals may be filtered by applying one or more bandpass filters to filter the EEG signals based on delta, theta, alpha and beta frequency bands. The EEG signals may be filtered into multiple frequency bands. In one example the channels may be channels associated with epistemic cognition comprise frontal region channels.

In one example, the spatial filtering selects channels that are associated with epistemic cognition.

In one example the spatial filtering selects channels F3, F4, F7, F8, AF3 and AF4, wherein channels F3, F4, F7, F8, AF3 and AF4 are associated with epistemic cognition.

The feature embedding module 416 is configured to project the preprocessed EEG data into a high dimensional feature space via convolution embedding layer. This output is concatenated with categorical variable embeddings, integrating both educational data and EEG signals into a unified representation of size d_{model}. The data is segmented into T′ fixed-length epochs using a sliding window technique, with T′≈T for large T, ensuring effective temporal representation for downstream processing.

The positional encoding module 418 is configured to add sinusoidal positional encodings to the embedded features to retain temporal context by unique positional information. The positional encoding module 418 is an optional module.

The final output of the Input Representation Block 410 is a C×T×d_{model} tensor, where each time step and channel is enriched with both signal features and positional context.

The transformer encoder block 420 is located after the input representation block 410. The transformer encoder block 420 is arranged in communication with the input representation block 410. The transformer encoder block 420 is configured to implement a transformer encoder. The transformer encoder is configured to capture local and long range temporal dependencies in the EEG signals. The transformer encoder block 420 is configured to refine feature representations over a plurality of iterations. In one example, the transformer encoder block 420 may implement a generic transformer encoder.

The transformer encoder block 420 comprises a multi-head attention layer 422. This layer computes contextual relationships between all time steps in the sequence. Each attention head processes a subset of the feature space. For a given query (Q), key (K), and value (V),

$$Attention ( Q , K , V ) = softmax ( \frac{Q {K}^{T}}{\sqrt{{d}_{k}}} ) V \qquad ( 1 )$$

where Q, K, V∈ [symbol] and dₖ is the dimension of the key vectors. The attention output is followed by a dropout layer for regularization.

The transformer encoder block 420 comprises a feed forward network (FFN) 422 is component operatively coupled to the multi head attention layer 422. The FFN 422 comprising a plurality of convolution layers and at least one dropout layer. The FFN layer 422 refines the attention output through two convolutional layers, with a dropout layer in between. The first layer projects features to a ff_dim-dimensional space with ReLU activation, and the second projects them back to the original dimensionality.

The transformer 400 may further comprise residual connections indicated as (Add & Norm). These residual connections improve gradient flow and preserve input information. The final output is the sum of the normalized feed-forward output and the residual connection from the attention step.

The Transformer Encoder Block 420 iteratively refines the feature representation over N iterations by attending to temporal patterns and enhancing contextual relationships. This enables the model to progressively capture hierarchical temporal features while preserving the spatial significance of EEG channels.

The transformer 400 further comprises a global average pooling block 430 located after the transformer encoder block 420 and arranged in communication with the transformer encoder block 420. The global average pooling block 430 is configured to reduce sequence length by averaging the output of the transformer encoder block across a temporal dimension into a single value per feature channel.

The global average pooling block 430 reduces the sequence length by averaging across the temporal dimension of the representation resulted from the previous block into a single value per feature channel:

$${X}_{G A P} ( c ) = \frac{1}{T} {\sum }_{t = 1}^{T} {X}_{c} ( t ) \qquad ( 2 )$$

where X_{GAP}(c) is the pooled feature for the channel c. The output is a feature vector with dimensionality equal to the number of channels. GAP minimizes computational overhead while preserving essential temporal information.

The transformer 400 comprises a classification block 440 positioned after the global average pooling block 430.

The classification block 440 may be a generic classifier and is the final stage of the transformer architecture 400. This final stage translates features from the Global Average Pooling Block into class predictions.

The classification block 440 is arranged in communication with the global average pooling block 430. The classification block 440 is configured to translate features from the global average pooling block into predictions of epistemic cognition states. Additionally, the classification block 440 is configured to classify epistemic cognition states based on the predictions and output the classified epistemic cognition states.

The classification block 440 may function as a decoder portion of the transformer 400. In the example shown in FIG. 4, the classification block 400 may comprise a Multi-Layer Perceptron (MLP) 442, a dropout regularization module 444 and an output layer 446.

The MLP 442 is configured to process the feature vector through fully connected (Dense) layers with ReLU activation, enabling the model to capture nonlinear relationships. The layer dimensions are defined by the mlp_units parameter, allowing flexibility in architecture design.

The dropout regularization module 444 is configured to deactivate one or more neurones in the MLP to prevent overfitting. The module 444 is controlled by the mlp_dropout parameter and deactivates a fraction of neurons after each dense layer to prevent overfitting and improve generalization.

The output layer 446 is configured to output a probability score for each class, wherein the probability score indicating the classification of a epistemic cognition state. The output layer may be a dense layer that produces the class probabilities. For binary classification, this layer contains a single neuron with a softmax activation function. The output is a probability score for each class (e.g., “understand” or “not understand”), with the class having the highest probability selected as the prediction.

Overall, the Classification Block 446 takes the output from the feature extraction layers, refines it through fully connected layers, and produces the final class probabilities. By combining MLP layers with dropout regularization and a softmax output, this block ensures robust predictions for the binary classification task.

The model 400 may be implemented as executable instructions or as an executable computer program, which can be executed by a computing apparatus 200, in particular the processor 202. The model 400 (i.e., the transformer based EEG classifier) may be written as a computer program in any appropriate software or programming language such as for example Python.

The model 400 i.e., the transformer based EEG classifier 400 was tested by conducting cross subject experiments and three datasets. The three datasets were publicly available EEG datasets collected during epistemic cognitive tasks. These datasets were used in testing to evaluate the proposed transformer based classifier 400. The datasets encompass diverse educational contexts, participant characteristics, and EEG recording setups. This offers a robust benchmark for validating the model's performance. FIG. 5 illustrates a table 500 that summarises the key attributes of the datasets.

Dataset 1 502 includes EEG data of computer science undergraduate students' comprehension of programming language e.g. Python programs. The EEG signals were recorded using a 32 electrode Biosemi system configured according to the international 10-20 system. Data were pre-processed with a 0.01-30 Hz bandpass filter and sampled at 200 Hz.

Dataset 2 504 is directed to EEG data from students understanding distance learning. Participants were from diverse backgrounds. EEG data was collected using the Emotiv Epoc X headset which features 14 channels. For each sensor five brain wave features Delta, Theta, Alpha, Beta and Gamma features were extracted.

Dataset 3 506 includes DDG signals collected from college students while watching MOOC videos tailored to align with or exceed their knowledge level. The data were acquired using a single channel wireless MindSet device which captured brain activity of the frontal lobe.

For testing the transformer 400 was implemented on a computing apparatus. In one example the transformer 400 was implemented with Tensorflow using Python 3.10. All experiments were conducted by the inventors on Google Colab with NVIDIA A100 (40 GB of GPU RAM).

The pre-processed samples (i.e., data from the three datasets) were divided into training and test sets according to an 80% to 20% ratio. The test samples were held out and used exclusively for the testing phase. Adam optimizer with a learning rate of 1×10-4 and batch size of 32 was used to train the network. During the training phase, a validation set was randomly extracted from the training set to assess the model convergence. The loss function was given by sparse_categorical_crossentropy, and model performance was evaluated by the accuracy. Finally, the trained model's performance was evaluated by the held-out testing samples. The transformer 400 and a standard model were evaluated with standard machine-learning evaluation metrics, namely accuracy, precision, recall, and F1 scores.

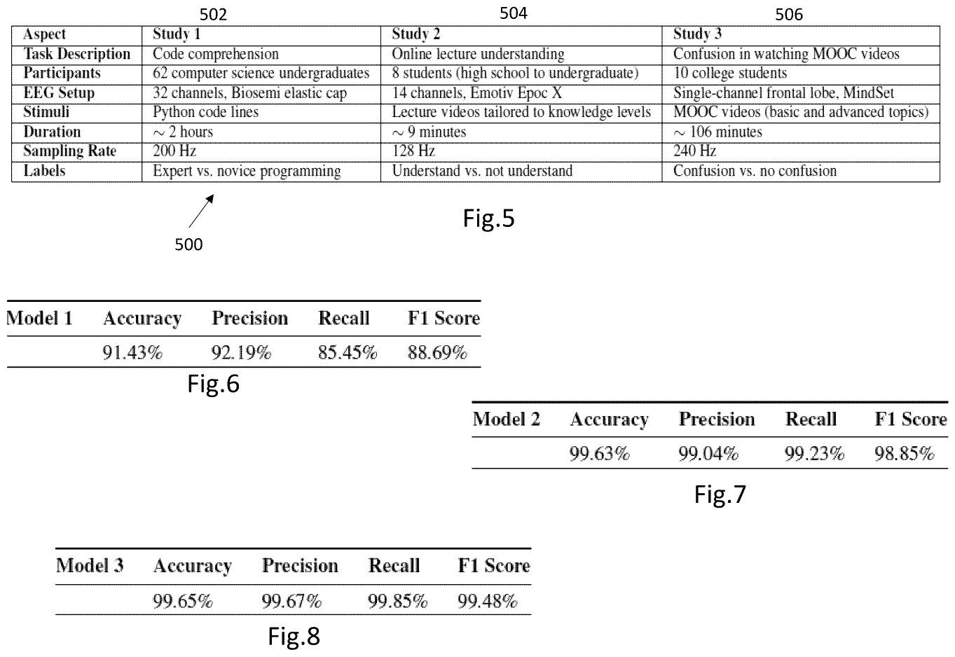

Three studies were conducted. In the first study the input to the model being tested consists of 17,268 samples each including 3 channel features, 1 categorical feature (gender) and a class label (novice=0, expert=1). The model successfully converged after 37 epochs. The test results are shown in FIG. 6. Model 1 refers to the model that was tested.

In the second study the input to the model consists of 68,831 samples each including 14 channel features (AF3, F7, F3, F5, T7, P7, O1, O2, P8, T8, FC6, F4, F8, AF4) filtered in alpha band (8-12 Hz), 1 educational feature (lesson ID) and 1 class label (subject understood). The model converged after 45 training epochs. The test results for the second study are shown in FIG. 7, where model 2 refers to the tested model.

In the third study the input to the model consists of 12,811 samples each including 8 EEG features (raw signals filtered into delta, theta, lower alpha, upper alpha, low beta, upper beta, lower gamma and upper gamma bands), 1 educational feature (Lesson ID) and 1 class label (a participant's subjective indicator of understanding). The model converged after 45 training epochs. The results are shown in FIG. 8. For each of study the model was optimized with a number of interacting hyperparameters.

The ECP layer 414 is advantageous because it processes the EEG signals i.e., EEG data for educational analysis ensuring optimal feature extraction. The ECP layer provides educational domain knowledge which leads to significant improvement in performance of the model. The inventors tested the effect of the ECP layer in improving model performance by conducting ablation experiments by comparing two model configurations. The “Original Model” refers to the complete proposed transformer architecture 400, as described in reference to FIG. 4. “Ablated models” are architectures with the specific functions of the ECP layer being removed, including temporal filtering, spatial filtering, spectral filtering and categorical embedding.

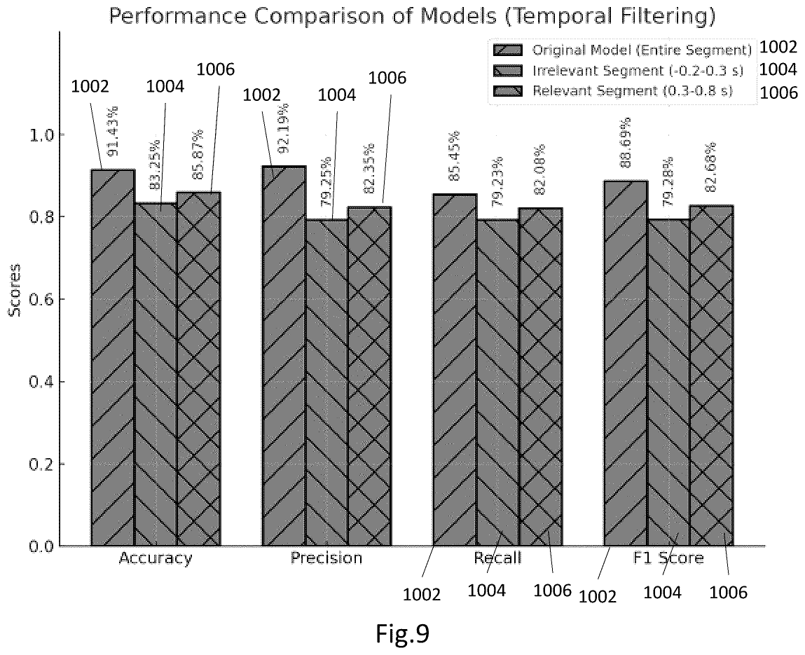

The first ablation experiment conducted by the inventors assessed the temporal filtering in the ECP layer 414 by comparing the original model using full EEG time series data with two ablation models. One ablation model having one restricted to irrelevant ERP segments and another ablation model being limited to relevant segments capturing N400 and P600 components. FIG. 9 illustrates a graph showing the performance comparison.

As shown in FIG. 9, the original model 1002 achieves the highest performance, with an accuracy of 91.43%, precision of 92.19%, recall of 85.45%, and F1 score of 88.69%. The relevant segment model 1004 shows a reduced performance, achieving an accuracy of 85.87%, precision of 82.35%, recall of 82.08%, and an F1 score of 82.68%. The irrelevant segment model 1006 performs the worst, with an accuracy of 83.25%, precision of 79.25%, recall of 79.23%, and an F1 score of 79.28%. These results highlight the limited utility of irrelevant time windows while demonstrating the advantage of leveraging the entire time series to capture comprehensive temporal features. Nevertheless, time windows associated with key cognitive events such as N400 and P600 facilitate the model's performance with less data. Overall, the results of FIG. 9 demonstrate the key role of temporal filtering in enhancing classification performance.

In the second ablation experiment the role of spatial filtering is assessed by analysing the effect of selecting different subsets of EEG channels on the model's performance. The original model utilizes data from all available EEG channels. Two ablation models are introduced: one using 6 less epistemic-relevant channels (T7, T8, FC5, FC6, and P7, P8 located in the temporal, frontal-central, and parietal regions for sensory, motor, and language processing) and another using 4 epistemic-relevant channels (F3, F4, F7, F8, AF3, and AF4 located in the frontal and anterior-frontal lobes critical for decision making, reasoning, and problem solving).

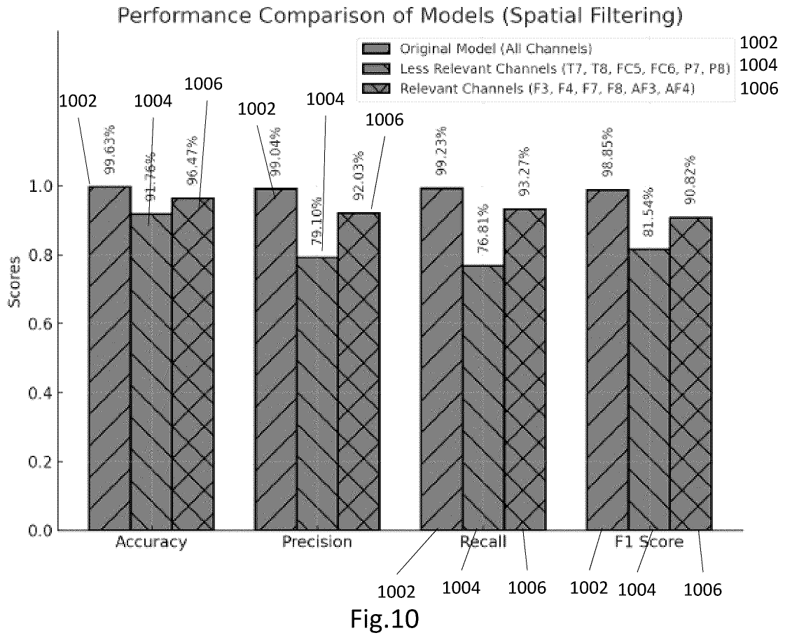

FIG. 10 illustrates the results of the second ablation experiment. As illustrated in FIG. 10, the original model 1002, which utilizes all available EEG channels, achieves the highest performance across all metrics, including an accuracy of 99.63%, a precision of 99.04%, a recall of 99.23%, and an F1 score of 98.85%. This demonstrates the benefit of leveraging all spatial information from EEG channels. In comparison, the ablation model 1004 using less relevant channels shows a substantial performance drop, with an accuracy of 91.76%, a precision of 79.10%, a recall of 76.81%, and an F1 score of 81.54%. Meanwhile, the ablation model 1006 using relevant channels maintains superior performance over the less relevant channel model, achieving an accuracy of 96.47%, a precision of 92.03%, a recall of 93.27%, and an F1 score of 90.82%. These results emphasize the importance of retaining spatially relevant EEG features, such as those associated with cognitive and decision-making processes, for optimal classification performance.

In the third ablation experiment the role spectral filtering is assessed. The original model uses all frequency bands (delta, theta, lower alpha, upper alpha, lower beta, upper beta, lower gamma, and upper gamma bands) extracted from the raw signal. In contrast, the first ablation model bypasses the filtering step and uses the raw EEG signal as input, while the second ablation model takes four EEG bands (theta, lower alpha, upper alpha, and lower beta bands) associated with learning, creativity, and thinking as input.

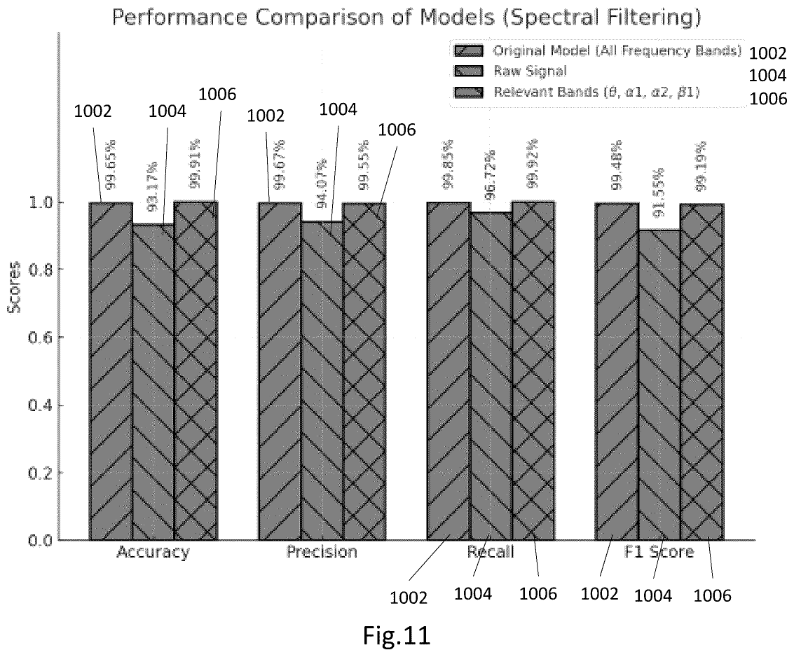

FIG. 11 illustrates the results of the third ablation experiment. As shown in FIG. 11, the original model 1002, which incorporates all frequency bands (delta, theta, alpha, beta, and gamma), achieves the highest performance across all metrics, with an accuracy of 99.65%, precision of 99.67%, recall of 99.85%, and F1 score of 99.48%. The second ablation model 1006, which uses relevant frequency bands (theta, lower alpha, upper alpha, and lower beta), performs comparably well, achieving an accuracy of 96.72%, precision of 99.55%, recall of 99.92%, and F1 score of 99.19%. In contrast, the first ablation model 1004, which uses raw EEG signals without spectral filtering, exhibits the lowest performance, with an accuracy of 93.17%, precision of 94.07%, recall of 99.91%, and F1 score of 91.55%. These results highlight the importance of spectral filtering in extracting task-relevant neural features and maximizing model performance, while also showing that even selected relevant bands can retain much of the model's efficacy compared to raw signal inputs.

The inventors conducted additional ablation experiments to assess the effectiveness of categorical embedding for gender and lesson. The performance of the original model 400 (with Gender ID or Lesson ID are categorical embeddings) was tested against an ablation model without these embeddings. The models were tested across four metrics: accuracy, precision, recall, and F1 score. In all cases, the inclusion of categorical embeddings through the ECP layer significantly enhances the models' performance.

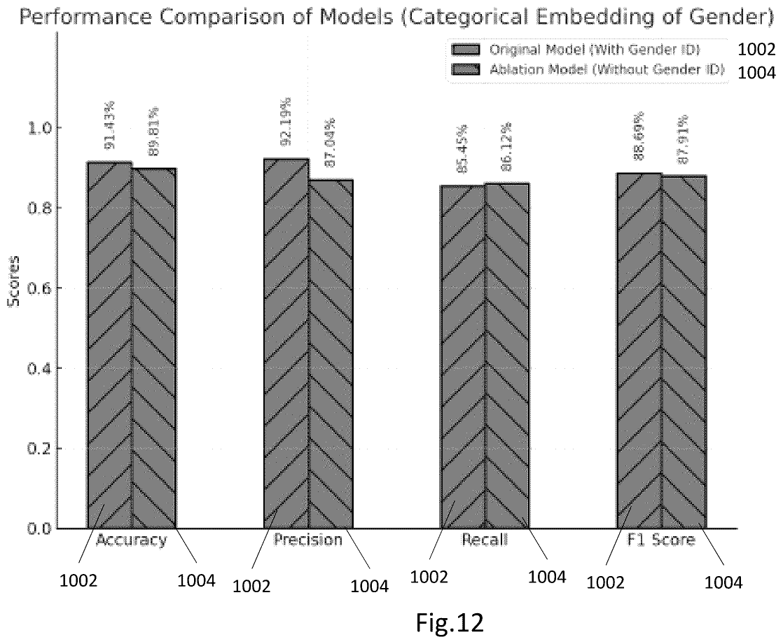

FIG. 12 illustrates the results of the first experiment of removing Gender ID. As shown in FIG. 12, removing Gender ID decreases the accuracy from 91.43% (with Gender ID model 1002) to 89.81% (without Gender ID model 1004), representing a decline of 1.77%. Similarly, the precision drops from 92.19% to 87.04%, showing a substantial decrease of 5.58%. These results underscore the importance of including gender information as a categorical embedding, particularly in enhancing recall and maintaining a balance between precision and recall. The model 400 (i.e., original model plot 1002) performs better due to the gender categorical embedding.

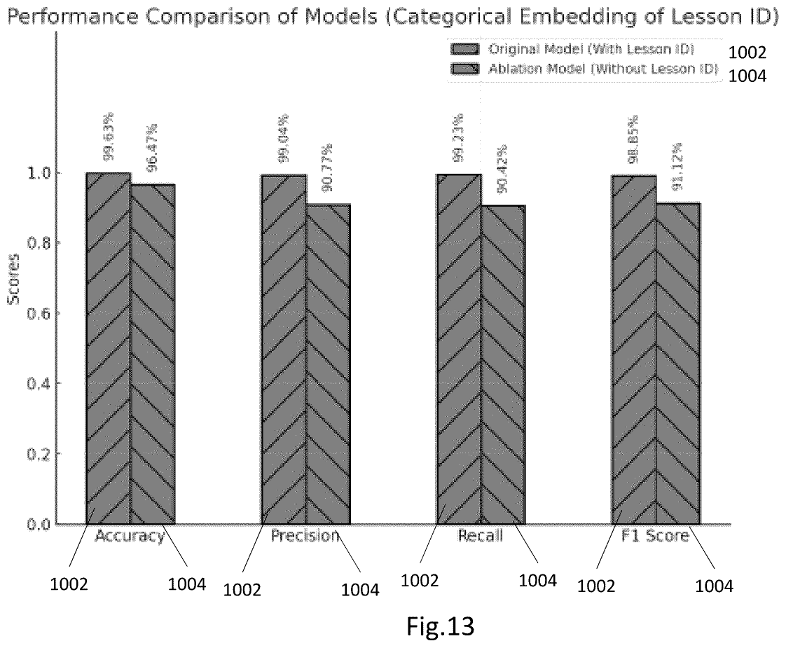

FIG. 13 illustrates the performance comparison of the original model (plot 1002) and ablation model (plot 1004) when the Lesson ID is removed. As shown in FIG. 13, excluding Lesson ID reduces the accuracy from 99.63% (with Lesson ID, plot 1002) to 96.47% (without Lesson ID, plot 1004), a decline of 3.17%, and precision from 99.04% to 90.77%, which is a notable decrease of 8.35%. These findings highlight the critical role of contextual information (encompassed in the Lesson ID) in capturing essential educational data and improving generalization. The results show the improved performance of model 400 since it includes categorical embeddings.

The experimental results demonstrate the superior performance in EEG classification tasks compared to state-of-the-art transformer based models, particularly in tasks associated with epistemic cognition. The results show the superior performance of the model 400. The experimental results demonstrate that transformer models are well suited for EEG classification for epistemic cognition. With capabilities such as hierarchical feature extraction, temporal attention, and spatial feature integration, transformers effectively address the complexities of EEG signals. These properties are particularly advantageous for educational studies that utilize EEG data.

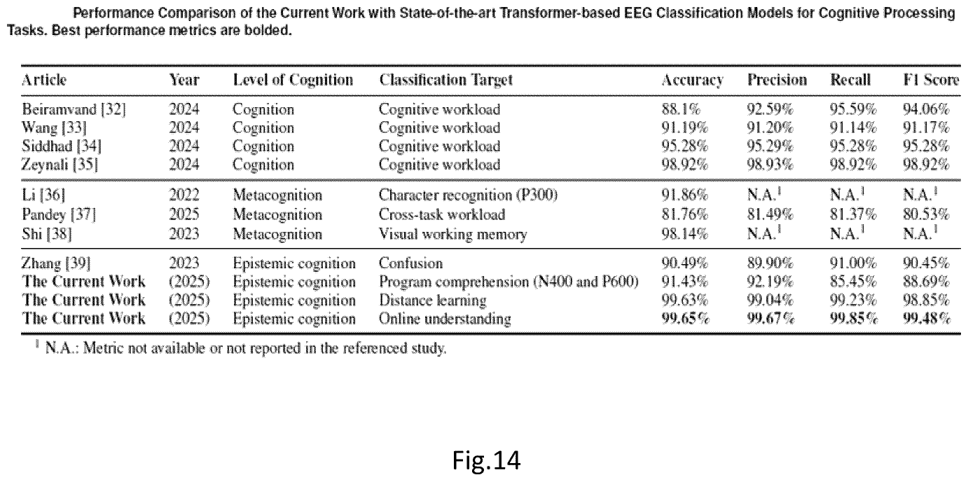

Additionally FIG. 14 illustrates the results of performance comparison of the current work (i.e., the system and method for classifying epistemic cognition states of a person from EEG signals in educational contexts) with state of the art transformer classifiers. FIG. 14 shows the performance of the current transformer-based EEG classifier 400 that includes an ECP layer as compared with state-of-the-art transformer classifiers. The performance of the current model is labelled “Current work” in the table of FIG. 14. As shown in FIG. 14, the current model performs better than the state-of-the-art models. The current model 400 is particularly performs better in classifying epistemic cognition states in online understanding and distance learning education contexts.

The integration of educational domain knowledge is technically achieved by the ECP layer 414. Its effects had been demonstrated by the ablation studies. These quantitative improvements shown in the experiments described above highlight the importance of educational domain knowledge in pre-processing, ensuring the extraction of meaningful and task relevant features. The ECP layer 414 further provides categorical embeddings which have a demonstrated impact on model performance, as shown in the experiments described above. These results suggest the important role of categorical embeddings in capturing essential contextual information during students' learning which leads to improved model performance and outputs. The model 400 achieved accuracy of 99.63% and 99.65% for distance learning and online understanding tasks, respectively, outperforming existing transformers in cognition and metacognition studies.

The system and method for classifying epistemic cognition states of a person from EEG signals in educational contexts, in particular the model 400 can enhance remote education through adaptive online content delivery systems. The system 100 is advantageous as it could be used to adjust the pace or complexity of lessons based on real-time student comprehension data, improving engagement and learning efficacy. Moreover, the model 400 integrates domain-specific features which further connects the above findings to teaching and learning scenarios. For example, the inclusion of categorical embeddings like Lesson ID significantly improves model accuracy, which showcases the importance of educational contextual data in refining predictions. Overall, the above insights can help inform the development of educational tools that combine neuroscientific evidence with targeted instructional design.

The EEG transformer 400 for epistemic cognition state classification is advantageous as it can enable real-time applications in classrooms and virtual learning environments. The model 400 maintains robust performance while significantly reducing computational overhead. This makes the model 400 more readily applicable in classrooms or other learning environments. The model 400 can play a role in modern education, enabling responsive and personalized learning experiences and provide educators with greater insights into students learning and highlight learning difficulties.

The described architecture of the model 400 is advantageous because it achieves state-of-the-art performance across varying educational contexts. By incorporating domain-specific preprocessing techniques (i.e., by incorporating educational domain knowledge), the model 400 is advantageous because it demonstrates outstanding classification results, emphasizing its applicability to real-world educational challenges.

The system 100 and method 300 of classifying epistemic cognition states is advantageous because it uses the model 400. The model 400 is also advantageous because it addresses the challenges and limitations such as scalability, interpretability, and generalizability to broader educational contexts. The described architecture provides a generalised model that can be applied in various educational contexts such as for example distance learning, program comprehension, online understanding etc. The model 400 also provides a lightweight and computationally efficient model as compared to using neural networks e.g., RNNs or CNNs.

Although not required, the embodiments described with reference to the Figures can be implemented as an application programming interface (API) or as a series of libraries for use by a developer or can be included within another software application, such as a terminal or personal computer operating system or a portable computing device operating system. Generally, as program modules include routines, programs, objects, components and data files assisting in the performance of particular functions, the skilled person will understand that the functionality of the software application may be distributed across a number of routines, objects or components to achieve the same functionality desired herein.

It will also be appreciated that where the methods and systems of the present invention are either wholly implemented by computing system or partly implemented by computing systems then any appropriate computing system architecture may be utilised. This will include stand alone computers, network computers and dedicated hardware devices. Where the terms “computing system” and “computing device” are used, these terms are intended to cover any appropriate arrangement of computer hardware capable of implementing the function described.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

Any reference to prior art contained herein is not to be taken as an admission that the information is common general knowledge, unless otherwise indicated.

The phrases ‘computer-readable medium’ or ‘machine-readable medium’ as used in this specification and claims should be taken to include, unless the context suggests otherwise, a single medium or multiple media. Examples of multiple media include a centralised or distributed database and/or associated caches. These multiple media store the one or more sets of computer executable instructions. The phrases ‘computer-readable medium’ or ‘machine-readable medium’ should also be taken to include any medium that is capable of storing, encoding or carrying a set of instructions for execution by a processor of a computing device and that cause the processor to perform any one or more of the methods described herein. The computer-readable medium is also capable of storing, encoding or carrying data structures used by or associated with these sets of instructions. The phrases ‘computer-readable medium’ and ‘machine readable medium’ include, but are not limited to, portable to fixed storage devices, solid-state memories, optical media or optical storage devices, magnetic media, and/or various other mediums capable of storing, containing or carrying instruction(s) and/or data. The ‘computer-readable medium’ or ‘machine-readable medium’ may be non-transitory.

Also, it is noted that the embodiments may be described as a process that is depicted as a flowchart, a flow diagram, a structure diagram, or a block diagram. Although a flowchart may describe the operations as a sequential process, many of the operations can be performed in parallel or concurrently. In addition, the order of the operations may be rearranged. A process is terminated when its operations are completed. A process may correspond to a method, a function, a procedure, a subroutine, a subprogram, etc., in a computer program. When a process corresponds to a function, its termination corresponds to a return of the function to the calling function or a main function.

Aspects of the systems and methods described above may be operable or implemented on any type of specific-purpose or special computer, or any machine or computer or server or electronic device with a microprocessor, processor, microcontroller, programmable controller, or the like, or a cloud-based platform or other network of processors and/or servers, whether local or remote, or any combination of such devices.

Furthermore, embodiments may be implemented by hardware, software, firmware, middleware, microcode, or any combination thereof. When implemented in software, firmware, middleware or microcode, the program code or code segments to perform the necessary tasks may be stored in a machine-readable medium such as a storage medium or other storage(s). A processor may perform the necessary tasks.

The methods or algorithms described in connection with the examples disclosed herein may be embodied directly in hardware, in a software module executable by a processor, or in a combination of both, in the form of processing unit, programming instructions, or other directions, and may be contained in a single device or distributed across multiple devices. A software module may reside in RAM memory, flash memory, ROM memory, EPROM memory, EEPROM memory, registers, hard disk, a removable disk, a CD-ROM, or any other form of storage medium known in the art. A storage medium may be coupled to the processor such that the processor can read information from, and write information to, the storage medium. In the alternative, the storage medium may be integral to the processor.

One or more of the components and functions illustrated the figures may be rearranged and/or combined into a single component or embodied in several components without departing from the scope of the invention. Additional elements or components may also be added without departing from the scope of the invention. Additionally, the features described herein may be implemented in software, hardware, as a business method, and/or combination thereof.

In its various aspects, embodiments of the invention can be embodied in a computer-implemented process, a machine (such as an electronic device, or a general-purpose computer or other device that provides a platform on which computer programs can be executed), processes performed by these machines, or an article of manufacture.

1. A computer-implemented method for classifying epistemic cognition states of a person in educational contexts comprising:
receiving an input dataset comprising EEG signals of a person,
modifying the EEG signals by integrating educational domain knowledge to extract features related to epistemic cognition states,
capturing local and long range temporal dependencies in the modified EEG signals,
averaging across the temporal dimension the temporal dependencies in the modified EEG signals,
classifying epistemic cognition states from the averaged temporal dependencies,
outputting the classified epistemic cognition states.

2. The method of claim 1, wherein modifying the EEG signals by integrating educational domain knowledge comprising:
bandpass filtering the EEG signals to isolate one or more predefined frequency bands,
spatial filtering the EEG signals to select channels associated with epistemic cognition,
temporal filtering the EEG signals to focus on signals with specific epistemic event windows.

3. The method of claim 2, wherein modifying the EEG signals comprising encoding categorical data as embeddings to incorporate contextual information into the modified EEG signals.

4. The method of claim 2, wherein the one or more predefined frequency bands have a frequency of between 0.01 Hz-30 Hz.

5. The method of claim 4, wherein the one or more predefined frequency bands are delta, theta, alpha and beta frequency bands.

6. The method of claim 3, wherein channels associated with epistemic cognition comprise frontal region channels.

7. The method of claim 6, wherein spatial filtering selects channels that are associated with epistemic cognition.

8. The method of claim 2, wherein modifying the EEG signals by integrating educational domain knowledge comprising: preprocessing the EEG signals, by applying independent component analysis to remove artefacts and normalize the EEG signals across channels to output two-dimensional multi-channel time series data.

9. The method of claim 8, wherein modifying the EEG signals by integrating educational domain knowledge comprising:
projecting the preprocessed EEG signals into a high dimensional feature space,
concatenating the projected preprocessed EEG signals with categorical variable embeddings, wherein the embeddings comprise integrating educational data and EEG signals into a unified representation, and;
segmenting the data into fixed length epochs by applying a sliding window technique.

10. The method of claim 9, comprising adding sinusoidal positional encodings into the embedded features or the fixed length epochs to retain temporal context based on unique positional information.

11. The method of claim 1, wherein the step of modifying the EEG signals by integrating educational domain knowledge results in a C×T×d_{model} tensor, where each time step and each channel comprises signal features and positional context, and wherein C represents the number of channels in the EEG signals, T represents the total time steps and d_{model} represents a unified representation that integrates EEG signals and educational data.

12. The method of method of claim 11, comprising iteratively refining feature representation over a predefined number of iterations by attending to temporal patterns and enhancing contextual relationships.

13. A machine learning model for classifying epistemic cognition states of a person in educational contexts, comprising:
an input representation block, the input representation block configured to transform raw EEG signals by incorporating educational domain knowledge to extract features for epistemic studies,
a transformer encoder block, the transformer encoder block positioned after the input representation block and arranged in communication with the transformer encoder block,
the transformer encoder block is adapted to receive the transformed EEG signals from the input representation block,
wherein the transformer encoder block configured to implement a transformer encoder that is adapted to capture local and long range temporal dependencies in the EEG signals, the transformer encoder block is configured to refine feature representations over a plurality of iterations,
a global average pooling block located after the transformer encoder block and arranged in communication with the transformer encoder block,
wherein the global average pooling block is configured to reduce sequence length by averaging the output of the transformer encoder block across a temporal dimension into a single value per feature channel,
a classification block located after the global average pooling block, the classification block is arranged in communication with the global average pooling block,
wherein the classification block is configured to translate features from the global average pooling block into predictions of epistemic cognition states, and;
wherein the classification block is configured to classify epistemic cognition states based on the predictions and output the classified epistemic cognition states.

14. The machine learning model of claim 13, wherein the machine learning model is a transformer-based EEG classifier,
wherein the input representation block comprising:
a data acquisition module configured to preprocess raw EEG signals using independent component analysis to remove artefacts and normalize the EEG signals across multiple channels, the data acquisition module configured to output a 2D multi-channel time series,
educational context preprocessing (ECP) module operatively coupled to the data acquisition module, the ECP module configured to:
receive the output of the data acquisition module,
apply a bandpass filter to the preprocessed EEG signals from the data acquisition module to isolate one or more predefined frequency bands, wherein the one or more predefined frequency bands are delta, theta, alpha and beta frequency bands,
spatially filter to select channels associated with epistemic cognitive processing, and;
temporal filter to focus on EEG signals within one or more epistemic event windows,
a feature embedding module operatively coupled to the ECP module, the feature embedding module configured to:
project the preprocessed EEG signals into a high dimensional feature space,
concatenate the projected signals with categorical variable embeddings thereby integrating educational data and EEG signals into a unified representation,
a positional encoding module operatively coupled to feature embedding module, the positional encoding module configured to add sinusoidal positional encodings to the embedded features,
wherein the transformer encoder block comprising:
a multi head attention layer configured to compute contextual relationships between all time steps in the sequence,
a feed forward network (FFN) component operatively coupled to the multi head attention layer, the FFN comprising a plurality of convolution layers and at least one dropout layer,
wherein the FFN configured to refine the attention output through the plurality of convolution layers and the one dropout layer,
wherein the classification block comprises:
a multi-layer perceptron (MLP) that comprises a plurality of fully connected layers, wherein the MLP is configured to receive a feature vector from the transformer encoder block and process the feature vector through the connected layers by with ReLU activation to capture non-linear relationships,
a dropout regularization module that is configured to deactivate one or more neurones in the MLP to prevent overfitting, and;
an output layer that is configured to output a probability score for each class, wherein the probability score indicating the classification of a epistemic cognition state.

15. A system for classifying epistemic cognition states of a person in educational contexts, comprising:
a computing apparatus comprising a processor and a memory unit being operatively coupled to each other,
the computing apparatus is configured to:
receive raw EEG signals,
process the raw EEG signals by applying a transformer-based EEG classifier to classify epistemic cognition states within the EEG signals,
wherein the transformer-based EEG classifier is configured to integrate educational domain knowledge into the raw EEG signals as part of the processing, and
output the classified epistemic cognition states.

16. The system of claim 15, wherein the computing apparatus is configured to:
receive an input dataset comprising EEG signals of a person,
modify the EEG signals by integrating educational domain knowledge to extract features related to epistemic cognition states,
capture local and long-range temporal dependencies in the modified EEG signals,
average across the temporal dimension the temporal dependencies in the modified EEG signals,
classify epistemic cognition states from the averaged temporal dependencies,
output the classified epistemic cognition states.

17. The system of claim 16, wherein when the transformer-based EEG classifier executed by the processor, the computing apparatus is further configured to:
bandpass filter the EEG signals to isolate one or more predefined frequency bands,
spatial filter the EEG signals to select channels associated with epistemic cognition,
temporal filter the EEG signals to focus on signals with specific epistemic event windows.

18. The system of claim 17, wherein:
the one or more predefined frequency bands are delta, theta, alpha and beta frequency bands,
the channels associated with epistemic cognition comprise frontal region channels and;
spatial filtering selects channels that are associated with epistemic cognition.

19. The system of claim 18, wherein the transformer-based EEG classifier comprises:
an input representation block configured to receive raw EEG signals and modify the EEG signals by integrating educational domain knowledge to extract features related to epistemic cognition states,
a transformer encoder block operatively coupled to the input representation block, wherein the transformer encoder block is positioned downstream to the input representation block,
wherein the transformer encoder block configured to capture local and long-range temporal dependencies in the modified EEG signals,
a global average pooling block operatively coupled to the transformer encoder block, wherein the global average pooling block being positioned downstream of the transformer encoder block,
wherein the global average pooling block configured to average across the temporal dimension the temporal dependencies in the modified EEG signals,
a classification block operatively coupled to the global average pooling block, the classification block positioned downstream of the global average pooling block,
wherein the classification block is configured to classify epistemic cognition states from the averaged temporal dependencies and output the classified epistemic cognition states.
